(19) United States
(12) Patent Application Publication
(10) Pub. No.: US 2019/0117066 A1
(43) Pub. Date: Apr. 25, 2019

(54) NON-INVASIVE TONOMETER FOR INTRAOCULAR PRESSURE MEASUREMENT AND TISSUE DUROMETER

(71) Applicant: Tarek Kurdy, Orlando, FL (US)

(72) Inventor: Tarek Kurdy, Orlando, FL (US)

(21) Appl. No.: 15/663,693

(22) Filed: Jul. 28, 2017

(51) Int. Cl. A61B 3/16; A61B 3/00; A61B 5/06; A61B 5/00

(52) U.S. Cl. CPC A61B 3/16; A61B 3/0041; A61B 5/068; A61B 5/7405; A61B 5/742; A61B 2503/40; A61B 5/6803; A61B 2562/0261; A61B 2562/0252; A61B 5/6821; A61B 5/6831; A61B 5/7455

(57) ABSTRACT

A tonometer for non-invasive measurement of intraocular pressure and or ocular durometer through a closed or open eyelid, and via sclera, includes a frame, a force sensor mounted with respect to the frame for measuring a force, a support structure mounted with respect to the frame, a position sensor mounted with respect to the frame, a movement mechanism, a contact tip, and a processing unit in communication with the force sensor and the position sensor, a memory unit, a display unit, a signaling mechanism, a power source, a power switch and a wireless communication module. A variant of the apparatus is also capable of functioning as a durometer measuring device to measure hardness of tissues such as wound tissue.

### RELATED PATENT APPLICATION AND INCORPORATION BY REFERENCE

This utility application claims the benefit under 35 USC 119(e) OF U.S. Provisional Patent Application No. 62/368,125 entitled “Non-Invasive Tonometer For Intraocular Pressure Measurement And Tissue Durometer”, filed Jul. 28, 2016 This related application is incorporated herein by reference and made a part of this application. If any conflict arises between the disclosure of the invention in this utility application and that in the related provisional application, the disclosure in this utility application shall govern. Moreover, any and all U.S. patents, U.S. patent applications, and other documents, hard copy or electronic, cited or referred to in this application are incorporated herein by reference and made a part of this application.

### BACKGROUND OF THE INVENTION

The subject matter described herein relates to a non-invasive tonometer device and a method of measuring intraocular pressure, and or ocular durometer of a subjects eye.

### SUMMARY OF THE INVENTION

Tonometers are known devices for detecting the intraocular pressure within an eye, as an indicator for Glaucoma, an eye disease. Most clinically available tonometers such as the Goldmann Tonometer and Tono-Pen (Reichert) are invasive, and measure intraocular pressure by making direct contact with the patient's cornea which can cause discomfort for the patient. This also requires the use of eye numbing medication which can cause irritation and can alter intraocular pressure. Additionally, measuring intraocular pressure through the cornea is known to be problematic as corneal thickness, shape and rigidity can vary from patient to patient. These devices are only available in a clinical setting, are expensive, not for home use, cannot be self administered, and are difficult to administer to children and infants. With the limitations of current devices being a factor, the majority of people with Glaucoma worldwide are unaware they have the disease.

The current method of eye specialists for the treatment of Glaucoma is to base treatments on just a few or less measurements of eye pressure throughout an extended period of time, such as a year. This is a problem because eye pressure can fluctuate not only within a year or a few months, but even within a day or an hour. 30% to 60% of Glaucoma sufferers experience diurnal spiking of eye pressure, meaning their eye pressure can rise dangerously, usually in the early morning hours, then return to a normal range throughout the day. These fluctuations can cause undetected damage to the optic nerve. With the limitations of current devices, diagnosis and monitoring of these patients is near impossible, as they present with normal pressures at doctor's visit during the day and will be unaware of the presence of disease until damage to optic nerve and vision loss occur. There is a growing trend within the field of Ophthalmology in which more and more eye specialists are realizing this and are recognizing the need for a non-invasive, home use glaucoma screening/monitoring device, capable of 24 hour monitoring to detect diurnal spiking and patient's individual eye pressure fluctuations in order to develop and deploy personalized and more effective treatment regimens.

A group of the population which would also benefit greatly from said device are survivors of blunt head trauma, such as car accident survivors or service men and service women experiencing head trauma. Head trauma and concussive blasts to the eye can result in the drainage system within the eye no longer functioning properly, leading to increased aqueous fluid buildup and Glaucoma. Having a non-invasive device that could be used in the field to quickly diagnose and monitor intraocular pressure and or ocular durometer would be a valuable tool for trauma victims.

Glaucoma is a complex eye disease most often associated with elevated intraocular pressure (normally above 20 mmhg) which can result in damage to the optic nerve and permanent vision loss. There are an estimated 100 million people worldwide who have Glaucoma. Most people who have the disease are unaware they have Glaucoma, as there are usually no noticeable symptoms associated with the disease other than vision loss. For this reason, it is important to have regular eye exams. Glaucoma is currently the leading cause of irreversible blindness. The World Health Organization estimates that 11 million people will experience blindness due to Glaucoma by 2020.

Durometer is a measurement for the hardness of a material. Ocular Durometer is a measurement of the hardness of the eye.

The Goldmann Applanation Tonometer (GAD is the most widely accepted and used tonometer in the Ophthalmology community and the accepted standard for measuring intraocular pressure. The GAT calculates intraocular pressure by relying on the Imbert Fick “Law”, which states that the pressure within a sphere (P) is roughly equal to an external force (F) needed to flatten a portion of the sphere divided by the area (A) of the sphere which is flattened:

P=F/A

Imbert Fick also assumes that:
The eye is a perfect sphere
With infinitely thin boundary
Boundary has no rigidity
   The problem with relying on this method is that the above assumptions are incorrect. The eye is a complex organ, not a perfect sphere, and has a rigid boundary (Scleral tissue) with known thickness. Despite being the most widely accepted method of measuring intraocular pressure today, this method is less than ideal. Proposed is an alternative method of diagnosing and monitoring Glaucoma by measuring directly the hardness or “ocular durometer” of the eye. The ocular durometer is a product of the force (F), measured by a force sensor resulting from indentation of the object (Eye) with durometer (D) by a preset distance (X).

F=DX

A first broad aspect provided herein is an apparatus for non-invasive measurement of intraocular pressure and or ocular durometer of an eye through an eyelid of a subject, wherein the apparatus comprises: a frame; a movement mechanism mounted with respect to the frame; a support structure mounted with respect to the frame; a force sensor mounted with respect to the movement mechanism; a contact tip mounted with respect to the force sensor; a dipole magnet mounted with respect to the force sensor and the movement mechanism; a position sensor mounted with respect to the frame; a microprocessor comprising a memory; a display unit; a power source; wherein the position sensor is configured to track and report the position of the contact tip to the microprocessor, wherein the support structure holds the eye in place, preventing any movement of the eye during measurement and also provides compression of orbital tissues, wherein the contact tip is placed against the eyelid of the same eye of the subject and moved further towards the eye until a predetermined force threshold is obtained by the force sensor and the correlated position of the contact tip is recorded as the “Zero Point” in the memory, wherein the contact tip is further moved a preset measurement distance towards the eye to obtain a measurement force and the force sensor reports said measurement force to the microprocessor, and wherein said measurement force is converted to an intraocular pressure and or durometer, then shown on the display unit and stored in the memory.

In an embodiment, the apparatus further comprises a communication unit for transmitting said measurement force or intraocular pressure to a remote receiver.

In an embodiment, the apparatus further comprises a memory card slot configured for a memory card capable of recording and storing data associated with measurement of the intraocular pressure and or durometer.

In an embodiment, the force sensor comprises: a strain gauge; a transducer; and a load cell.

In an embodiment, the position sensor comprises: a magnetic position sensor; an optical position sensor; a capacitive position sensor; and a linear variable differential transformer.

In an embodiment, the movement mechanism comprises: a slide mechanism; a roller mechanism; a dovetail rack mechanism; a rack and pinion mechanism; a screw mechanism; a belt-drive mechanism; a manually driven mechanism; a motorized mechanism; and a maglev system.

In an embodiment, the signaling mechanism comprises: an audible signal; a visual signal; and a tactile signal.

In an embodiment, the power source comprises: an alternating current; a direct current; and a solar cell.

In an embodiment, the communication unit comprises: a wireless device; and a wired device.

In an embodiment, the first predetermined force threshold is at least 1.00 gram.

In an embodiment, the first predetermined force threshold is about 1.00 gram.

In an embodiment, the first predetermined force threshold is within a range of ±20% of 1.00 gram.

In an embodiment, the preset measurement distance to obtain a second measurement force is about 0.01 inch.

In an embodiment, the preset measurement distance to obtain a second measurement force is within a range of about ±20% of 0.01 inch.

In an embodiment, the intraocular pressure measurement is not dependent on time.

In an embodiment, the sampling rate for the force sensor by the microprocessor is at least 1000 Hz.

In an embodiment, the sampling rate for the force sensor by the microprocessor is about 1000 Hz.

In an embodiment, the sampling rate for the force sensor by the microprocessor is within a range of about ±20% of 1000 Hz.

In an embodiment, the force sensor requires only one measurement force acquired over one predetermined measurement distance to obtain a suitable intraocular pressure measurement.

In an embodiment, the contact tip is manually moved quickly or slowly to determine sufficient intraocular pressure and or ocular durometer reading.

In an embodiment, the contact tip is moved in an automated fashion by a micro step motor or servo motor.

In an embodiment, where measurement is taken through front or corner of eyes.

In an embodiment, where measurement is taken through the eyelid when the eyelid is open or closed.

In an embodiment, the apparatus is configured to assist an individual in the diagnosis of Glaucoma.

In an embodiment, the apparatus is configured for use by an individual comprising: a medical clinician; a medical technician; a trained individual; an untrained individual; and a patient.

In an embodiment, the apparatus is administered by one individual to another individual or administered by one's self to one's self.

In an embodiment, diagnosis and monitoring of Glaucoma comprises: Glaucoma in adults and Glaucoma in infants and young children.

In an embodiment, the apparatus is configured for diagnosis and monitoring of Glaucoma in an animal.

In an embodiment, the animal comprises: a human; a horse; a dog; and a cat.

In an embodiment, the apparatus is configured for use by an individual in a setting comprising: in a medical professional setting; a home setting; an ambulatory setting; a field setting, and a combat setting.

In an embodiment, the apparatus further comprises a wireless adapter configured to connect with a mobile device and further configured to receive said second measurement force, intraocular pressure and or durometer from the apparatus without a need to pair said apparatus with said mobile device.

A second broad aspect provided herein is an apparatus for non-invasive measurement of intraocular pressure and or durometer of an eyelid and ocular durometer through an eyelid and via sclera of a subject, capable of 24 hour monitoring of intraocular pressure and or ocular durometer, the apparatus comprising: a frame; a movement mechanism mounted with respect to the frame; a support structure mounted with respect to the frame; a force sensor mounted with respect to the movement mechanism; a contact tip mounted with respect to the force sensor; a dipole magnet mounted with respect to the movement mechanism and frame; a position sensor mounted with respect to the frame; a computer implemented system comprising: a digital processing device comprising an operating system configured to perform executable instructions and a memory; a computer program including instructions executable by the digital processing device to generate an intraocular pressure and or ocular durometer measurement comprising: a software module configured to calculate an intraocular pressure and or ocular durometer based on a measurement force obtained from the force sensor; a display unit; a power source; wherein the position sensor is configured to track and report the position of the contact tip to the digital processing device; wherein the support structure holds the eye in place during measurement, preventing the eye from moving, and provides compression to orbital tissues, to prevent interference in eye measurement, wherein the contact tip is placed against the eyelid of the same eye of the subject and moved towards the eye until a predetermined force threshold is obtained by the force sensor and the correlated position of the contact tip is recorded as the “Zero Point” in the memory, wherein the contact tip is further moved a preset measurement distance towards the eye to obtain a second measurement force and the force sensor reports said second measurement force to the digital processing device, and wherein said second measurement force is converted to an intraocular pressure and or ocular durometer, stored in the memory and showed on the display.

In an embodiment, the force sensor comprises: a strain gauge; a transducer; and a load cell.

In an embodiment, the position sensor comprises: a magnetic position sensor; an optical position sensor; a capacitive position sensor; and a linear variable differential transformer.

In an embodiment, the movement mechanism comprises: a slide mechanism; a roller mechanism; a dovetail rack mechanism; a rack and pinion mechanism; a screw mechanism; a belt-drive mechanism; a manually driven mechanism; a motorized mechanism; and a maglev system.

In an embodiment, the signaling mechanism comprises: an audible signal; a visual signal; and a tactile signal.

In an embodiment, the power source comprises: an alternating current; a direct current; and a solar cell.

In an embodiment, the communication unit comprises: a wireless device; and a wired device.

In a third broad aspect provided herein is a computer-implemented method of non-invasive measurement of intraocular pressure and or ocular durometer of an eye through an eyelid of a subject, the method comprises: operating an apparatus comprising: a frame; a movement mechanism mounted with respect to the frame; a support structure mounted with respect to the frame; a force sensor mounted with respect to the movement mechanism; a contact tip mounted with respect to the force sensor; a dipole magnet mounted with respect to the frame and movement mechanism; a position sensor mounted with respect to the frame; a power source; and a microprocessor comprising a memory; monitoring, by the position sensor, the position of the contact tip and reporting said position to the microprocessor; placing the support structure around an eye, preventing the eye from moving and the contact tip against an eyelid of the same eye of the subject, moving the contact tip towards the eye until a predetermined force threshold is obtained by the force sensor; recording the correlated position of the contact tip as the “Zero Point” in the memory; causing the contact tip to move further a preset distance into the eyelid of the subject towards the eye and obtaining a second measurement force; recording the second measurement force in the memory; causing the microprocessor to calculate an intraocular pressure and or ocular durometer based on the second measurement force obtained from the force sensor; displaying said intraocular pressure and or ocular durometer on the display and storing said intraocular pressure and or ocular durometer in the memory.

In an embodiment, the force sensor comprises: a strain gauge; a transducer; and a load cell.

In an embodiment, the position sensor comprises: a magnetic position sensor; an optical position sensor; a capacitive position sensor; and a linear variable differential transformer.

In an embodiment, the movement mechanism comprises: a slide mechanism; a roller mechanism; a dovetail rack mechanism; a rack and pinion mechanism; a screw mechanism; a belt-drive mechanism; a manually driven mechanism; a motorized mechanism; and a maglev system.

In an embodiment, the signaling mechanism comprises: audible signaling; visual signaling; and tactile signaling.

In an embodiment, the power source comprises: an alternating current; a direct current; and a solar cell.

In an embodiment, the apparatus further comprises a communication unit for transmitting said measurement force and or intraocular pressure to a remote receiver.

In an embodiment, the communication unit comprises: a wireless device; and a wired device.

In an embodiment, the apparatus further comprises a memory card slot configured for a memory card capable of recording and storing data associated with measurement of the intraocular pressure.

A fourth broad aspect provided herein is an apparatus for non-invasive measurement of intraocular pressure and or ocular durometer of an animal such as horse, cat or dog, the apparatus comprises: a frame; a movement mechanism mounted with respect to the frame; a support structure mounted with respect to the frame; a force sensor mounted with respect to the movement mechanism; a contact tip mounted with respect to the force sensor; a dipole magnet mounted with respect to the frame and movement mechanism; a position sensor mounted with respect to the frame; a microprocessor comprising a memory; a display unit; a power source; wherein the position sensor is configured to track and report the position of the contact tip to the microprocessor; wherein the support structure is placed on one side of a tissue mass and the contact tip is placed against another side of the tissue mass of a subject, wherein said contact tip is moved towards the tissue mass until a predetermined force threshold is obtained by the force sensor, and the correlated position of the contact tip is recorded as the “Zero Point” in the memory, wherein the contact tip is further moved a preset measurement distance towards the tissue mass to obtain a second measurement force and the force sensor reports said measurement force to the microprocessor, and wherein said second measurement force is converted to a tissue durometer measurement, is shown on the display and stored in the memory.

In an embodiment, the apparatus further comprises a communication unit for transmitting said second measurement force and or durometer to a remote receiver.

In an embodiment, the communication unit comprises: a wireless device; and a wired device.

In an embodiment, the apparatus further comprises a memory card slot configured for a memory card capable of recording and storing data associated with measurement of the durometer.

In an embodiment, the animal is a human of any age.

In an embodiment, the tissue comprises: ocular tissues; wound tissue; osteo tissues; dermal tissues; venal tissues; or viscero tissues.

In an embodiment, the predetermined force threshold is at least 1.00 gram.

In an embodiment, the predetermined force threshold is about 1.00 gram.

In an embodiment, the predetermined force threshold is within a range of ±20% of 1.00 gram.

In an embodiment, the preset measurement distance to obtain a measurement force is about 0.01 inch.

In an embodiment, the preset measurement distance to obtain a second measurement force is within a range of about ±20% of 0.01 inch.

In an embodiment, the durometer measurement is not dependent on time.

In an embodiment, the sampling rate of the force sensor is at least 1000 Hz.

In an embodiment, the sampling rate of the force sensor is about 1000 Hz.

In an embodiment, the sampling rate of the force sensor is within a range of ±20% of 1200 Hz.

In an embodiment, the force sensor requires only one measurement force acquired over one predetermined measurement distance to obtain a suitable durometer measurement.

In an embodiment of any of the above apparatuses, wherein the contact tip comprises a plurality of probe tip geometries suitable for contacting a tissue surface without causing detrimental or naturally reversing penetration.

In an embodiment of any of the above apparatuses, the device further comprises an audible signal device configured to alert a user when a measurement is completed.

In an embodiment of the method described above, the operating step is performed by: medical professional; a patient (one's self); or a non-medical professional.

A fifth broad aspect provided herein is an apparatus for non-invasive measurement of wound tissue durometer of a subject, the apparatus comprising: a support structure configured to hold wound tissue in place; a movement mechanism mounted with respect to the frame; a force sensor mounted with respect to the movement mechanism; a contact tip mounted with respect to the force sensor; a dipole magnet mounted with respect to the frame and the movement mechanism; a position sensor mounted with respect to the frame; a microprocessor comprising a memory; a display unit; a power source; wherein the position sensor is configured to track and report the position of the contact tip to the microprocessor, wherein the support structure is configured to prevent movement of the wound tissue of the subject, wherein the contact tip is further pressed against the wound tissue until a predetermined force threshold is obtained by the force sensor and the correlated position of the contact tip is recorded as the “Zero Point” in the memory, wherein the contact tip is further moved a preset measurement distance into the wound tissue to obtain a second measurement force and the force sensor reports said second measurement force to the microprocessor, and wherein said second measurement force is converted to a wound tissue durometer, then shown on the display unit and stored in the memory.

In some embodiments, the apparatus further comprises a communication unit for transmitting said measurement force and or wound tissue durometer to a remote receiver.

In some embodiments, the apparatus further comprises, a memory card slot configured for a memory card capable of recording and storing data associated with measurement of the wound tissue durometer.

In some embodiments, the force sensor comprises: a strain gauge; a transducer; and a load cell.

In some embodiments, the position sensor comprises: a magnetic position sensor; an optical position sensor; a capacitive position sensor; and a linear variable differential transformer.

In some embodiments, the movement mechanism comprises: a slide mechanism; a roller mechanism; a dovetail rack mechanism; a rack and pinion mechanism; a screw mechanism; a belt-drive mechanism; a manually driven mechanism; a motorized mechanism; and a maglev system.

In some embodiments, the signaling mechanism comprises: audible signaling; visual signaling; and tactile signaling.

In some embodiments, the power source comprises: an alternating current; a direct current; and a solar cell.

In some embodiments, the communication unit comprises: a wireless device; and a wired device.

In some embodiments, the support structure comprises an eye cup.

In some embodiments, the opening in the frame is: positioned above the cornea of the eye of the subject; positioned below the cornea of the eye of the subject; positioned to the left of the cornea of the eye of the subject; and positioned to the right of the cornea of the eye of the subject.

In some embodiments, the first predetermined force threshold is at least 1.00 gram.

In some embodiments, the first predetermined force threshold is about 1.00 gram.

In some embodiments, the first predetermined force threshold is within a range of ±20% of 1.00 gram.

In some embodiments, the preset measurement distance to obtain a second measurement force is about 0.01 inch.

In some embodiments, the preset measurement distance to obtain a second measurement force is within a range of about ±20% of 0.01 inch.

In some embodiments, the intraocular pressure measurement is not dependent on time.

In some embodiments, the sampling rate for the force sensor by the microprocessor is at least 1000 Hz.

In some embodiments, the sampling rate for the force sensor by the microprocessor is about 1000 Hz.

In some embodiments, the sampling rate for the force sensor by the microprocessor is within a range of about ±20% of 1000 Hz.

In some embodiments, the force sensor requires only one measurement force acquired over one predetermined measurement distance to obtain a suitable intraocular pressure measurement.

In some embodiments, the measurement is taken through the eyelid in a region not covering the cornea.

In some embodiments, the apparatus is configured to assist an individual in the diagnosis of Glaucoma.

In some embodiments, the apparatus is configured for diagnosis and monitoring of Glaucoma.

In some embodiments, the apparatus is configured for use on human subjects of any age.

In some embodiments, the apparatus further comprises a wireless adapter configured to connect with a mobile device and further configured to receive said second measurement force or intraocular pressure from the apparatus without a need to pair said apparatus with said mobile device.

In some embodiments, the apparatus is configured for use by individuals comprising: medical clinicians; medical technicians; trained individuals; untrained individuals; and patients.

In some embodiments, the diagnosis and monitoring of Glaucoma comprises: Adult Glaucoma and Primary Juvenile Glaucoma.

In some embodiments, the animal comprises: a human; a horse; a dog; and a cat.

### BRIEF DESCRIPTION OF THE DRAWINGS

FIG. 1 displays a cross sectioned side view of the device in use.

FIG. 2 shows a sample position-force diagram which details the measurement thresholds.

FIG. 3 depicts an isometric view of the support structure.

FIG. 4 depicts a 2nd embodiment in a night band for night and early morning monitoring of diurnal spiking

FIG. 5 depicts a 3rd embodiment for measuring wound tissue durometer

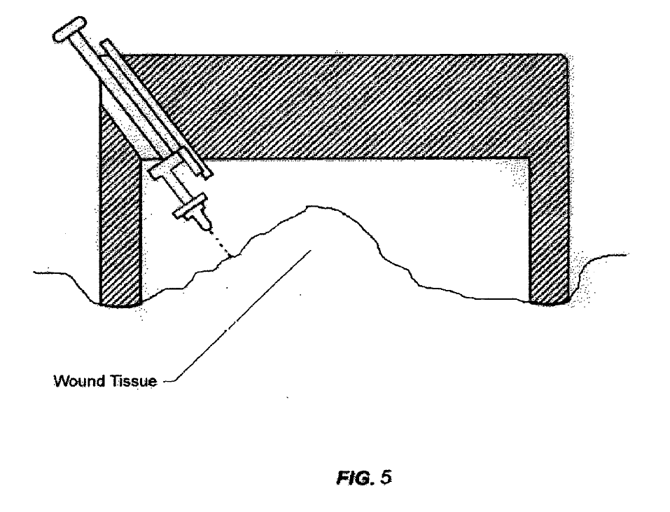

The novel features of the invention are set forth with particularity in the appended claims. A better understanding of the features and advantages of the present invention will be obtained by reference to the following detailed description that sets forth illustrative embodiments.

### DETAILED DESCRIPTION OF THE INVENTION

### Movement Mechanism

In some embodiments, the movement mechanism comprises: a slide mechanism; a roller mechanism; a dovetail rack mechanism; a rack and pinion mechanism; a screw mechanism; a belt-drive mechanism; a manually driven mechanism; a motorized mechanism; and a maglev system.

In some embodiments, a slide mechanism comprises a type of plain bearing.

In some embodiments, a roller mechanism comprises a non-motorized linear slide with a stationary linear base and a moving carriage.

In some embodiments, a dovetail rack mechanism comprises a non-motorized linear slide that contains a v-shaped stationary linear base and a correspondingly mating moving carriage.

In some embodiments, a rack and pinion mechanism comprises a form of linear actuator that contains a pair of gears which convert rotational motion into parallel linear motion.

In some embodiments, a screw mechanism comprises a type of linear actuator that contains a pair of gears which convert rotational motion into perpendicular linear motion.

In some embodiments, a belt-drive mechanism comprises an energy transmission device employing a loop of flexible material used to link two or more rotating shafts mechanically.

In some embodiments, a maglev system comprises a non-contact linear actuator that uses magnetic levitation to translate an object.

In still further embodiments the movement mechanism comprises: an elastic band; a spring; a weighted pulley; a pneumatic system; a hydraulic system; and a linear actuator.

### Force Sensor

In some embodiments, the force sensor comprises: a strain gauge; a transducer; and a load cell.

In some embodiments, a strain gauge comprises a device for indicating the strain of a material or structure at the point of attachment.

In some embodiments, a load cell comprises a transducer that is used to create an electrical signal whose magnitude is directly proportional to the force being measured.

In still further embodiments the force sensor comprises: a spring scale; a hydraulic load cell; and a pneumatic load cell.

### Position Sensor

In some embodiments, the position sensor comprises a magnetic position sensor; an optical position sensor; a capacitive position sensor; and a linear variable differential transformer.

In some embodiments, a magnetic position sensor comprises a type of Hall Effect Sensor, which emits a voltage in proportion to the proximity distance of a magnet.

In some embodiments, an optical position sensor comprises a device capable of measuring the position of a light spot in one or two-dimensions on a sensor surface.

In some embodiments, a capacitive position sensor comprises a non-contact sensor based of measured capacitance.

In some embodiments, a linear variable differential transformer comprises a type of electrical transformer used for measuring linear displacement.

In still further embodiments the position sensor comprises: a potentiometer; a rotary encoder; and a linear encoder.

### Signaling Mechanism

In some embodiments, the signaling mechanism comprises: an audible signal; a visual signal; and a tactile signal.

In some embodiments, the audible signaling mechanism comprises a variety of sounds or sound patterns.

In some embodiments, the visual signaling mechanism comprises: lights, flashing light patterns; colored lights; monitors; displays; and backlights.

In still further embodiments the tactile signaling comprises: a vibrator; an air jet; a prodder; and an electrical shocker.

### Contact Tip

In some embodiments, the device includes a contact tip which comprises a plurality of geometries suitable for contacting a tissue surface without causing detrimental or naturally reversing penetration.

In some embodiments, the contact tip's geometry is comprised of: a convex semisphere, which minimizes surface area with the eyelid as to minimize resistance from eyelid and focus on resistance force from eye, other embodiments a semicylinder; a flat plane; and a curved plane.

In some embodiments, the contact tip is comprised from a biologically safe and non-irritating material comprising: rubber; plastic; glass; and silicone.

### Microprocessor

In some embodiments, the device includes a microprocessor comprising: an ARM processor DSP; a Raspberry PI; Arduinos.

In some embodiments, software associated with such modules may reside in RAM memory, flash memory, ROM memory, EPROM memory, EEPROM memory, registers, a hard disk, a removable disk, a CD-ROM, or any other suitable form of storage medium known in the art.

In still further embodiments, the display comprises a combination of microprocessor devices such as those disclosed herein.

### Memory

In some embodiments, the device includes a memory device, card, or slot.

In some embodiments, the storage and/or memory device comprises one or more physical apparatuses used to store or accept data or programs on a temporary or permanent basis.

In other embodiments, the storage device comprises: CD-ROMs; DVDs; flash memory devices; magnetic disk drives; magnetic tapes drives; optical disk drives; and cloud computing based storage.

Display Unit

In some embodiments, the microprocessor communicates visual information to a user via a display unit.

In some embodiments, the display unit comprises: a cathode ray tube (CRT); a liquid crystal display (LCD); a thin film transistor liquid crystal display (TFT-LCD); a passive-matrix OLED display (PMOLED); an active-matrix OLED (AMOLED) display; a plasma display; and a video projector.

### Mobile Device

In some embodiments, the microprocessor is capable of communicating with a mobile device.

In some embodiments, a mobile device comprises: server computers; desktop computers; laptop computers; notebook computers; sub-notebook computers; netbook computers; netpad computers; set-top computers; media streaming devices; handheld computers; Internet appliances; mobile smartphones; tablet computers; personal digital assistants; video game consoles; smartphones; televisions; video players; digital music players; tablet computers; and vehicles.

### Software Module

In some embodiments, the device employs a software module configured to calculate an intraocular pressure based on a measurement force obtained from the force sensor.

In some embodiments, the software module comprises: Linux; Microsoft® Windows®; Apple® Mac OS X®; UNIX®; GNU/Linux®; Nokia® Symbian® OS; Apple® iOS®; Research In Motion® BlackBerry OS®; Google® Android®; Microsoft® Windows Phone® OS; Microsoft® Windows Mobile® OS; Linux®; and Palm® WebOS®.

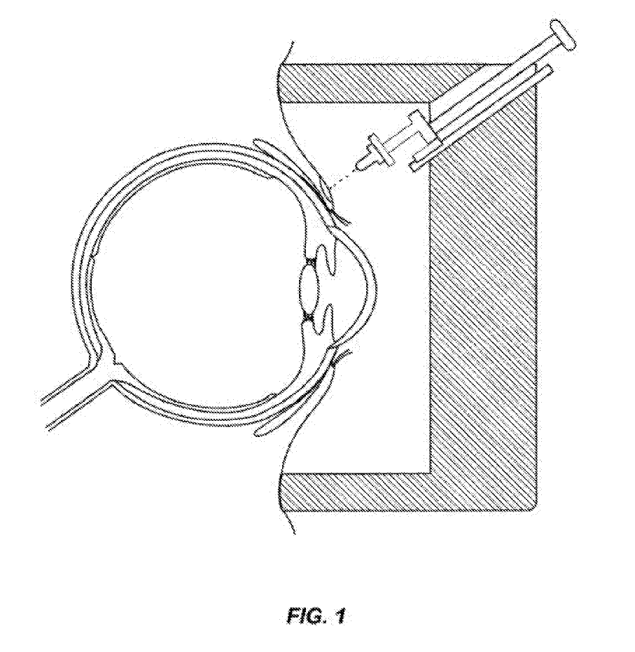

Per FIG. 1, there is seen a support structure 101, a frame 102, a contact tip 103, a force sensor 104, a movement mechanism 105, a dipole magnet 106, a position sensor 107 and an eyelid 108.

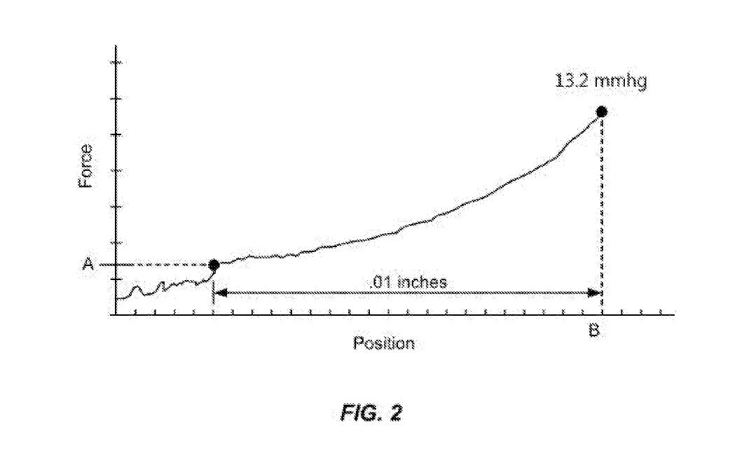

Per FIG. 2, there is seen a force threshold 201, a preset measurement distance 202, a first measurement point 203, a second measurement force 204, a second measurement point 205, a first position measurement 206 and a second position measurement 207.

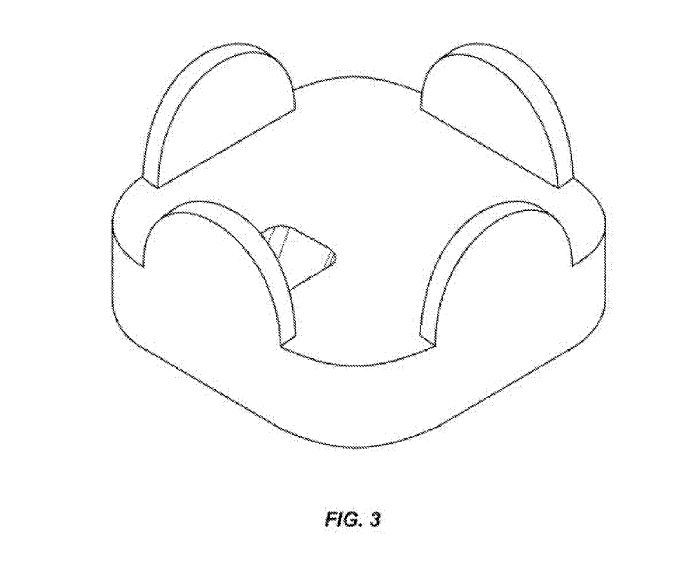

Per FIG. 3 there is seen a support structure 101 and a frame 102.

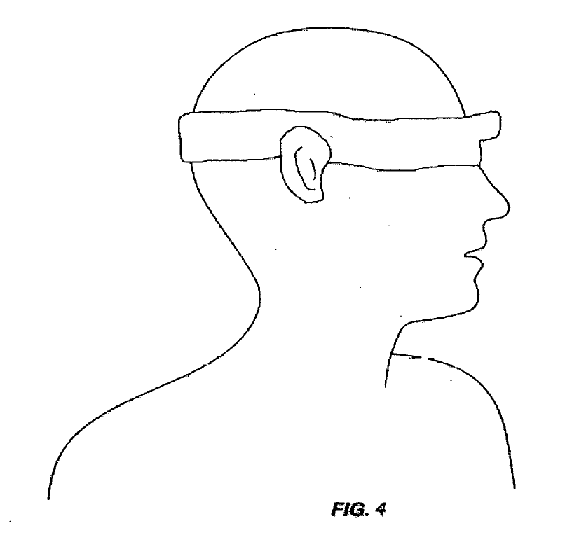

Per FIG. 4 there is seen an embodiment for 24 hour monitoring with preset measurement times.

In the first preferred mode of the device, as seen in FIG. 1, the support structure 101 contains a frame 102 oculus, to which the movement mechanism 105 and the position sensor 107 are mounted. The contact tip 103, which applies contact pressure to the eye directly or through the eyelid 108, the force sensor 104, which measures the force imparted on the eye, the dipole magnet 106, which enables translation measurement via the position sensor 107, and the plunger 109, which allows a practitioner to impart manual force on the eye through the device 10, are constrained to move along a single direction of freedom, towards and away from the eye, by the movement mechanism 105.

Per the chart displayed in FIG. 2, as the force applied to the eye increases and the eye begins to compress, the dipole magnet 106 moves towards the eye and the measured position value increases. Once the force sensor 104 detects an applied force above the force threshold 201, the first position measurement 206, of the dipole magnet 106 along the position sensor 107, is recorded by the microprocessor. Once the position sensor 107 detects that the dipole magnet 106 has translated a distance from the first position measurement 206 equal to the preset measurement distance 202, the microprocessor records the second position measurement 207 and the second measurement force 204 imparted through the contact tip 103 and the force sensor 104. The microprocessor can then calculate, store and transmit the patient's intraocular pressure, using the second measurement force 204, and alternatively any additional positions, translations and forces recorded through the procedure.

As used herein, and unless otherwise specified, the lower left face of the support structure 101, as displayed in in FIG. 3, shall be referred to as the “front face”, whereas the opposing, hidden face shall be referred to as the “back face”

As used herein, and unless otherwise specified, the distance between the front face and the back face shall be referred to as the “width” of the support structure 101

As used herein, and unless otherwise specified, the lower right face of the support structure 101, as displayed in in FIG. 3, shall be referred to as the “right face”, whereas the opposing, hidden face shall be referred to as the “left face”

As used herein, and unless otherwise specified, the distance between the right face and the left face shall be referred to as the “length” of the support structure 101

As used herein, and unless otherwise specified, the upper right face of the support structure 101, as displayed in in FIG. 3, shall be referred to as the “top face”, whereas the opposing, hidden face shall be referred to as the “bottom face”

Per FIG. 3, the top face of the support structure 101 contains one or more lobes which press and provide friction against the patient's face to steady the device 10 during use. In the preferred mode of the device 10 herein, the lobes are semicircular, but can alternatively be formed of any obtuse semi-polygon. The lobes should be positioned about the outer edges of the support structure 101 in a symmetrical radial array.

Additionally, the frame's 102 length should lie symmetrically within the length of the support structure 101, such that its upper aperture is located within the front half of the support structure 101 and such that its lower aperture pierces the front and bottom faces of the support structure 101. As such, the frame's 102 parallel front-facing and back-facing planes is within about 15° to about 75° relative to the top and bottom faces of the support structure 101.

In some embodiments, the angle between the frame's 102 parallel front-facing and back-facing planes, and the top and bottom faces of the support structure 101, is more than about 15°

In some embodiments, the angle between the frame's 102 parallel front-facing and back-facing planes, and the top and bottom faces of the support structure 101, is less than about 75°

In some embodiments, the contact tip's is comprised from a biologically safe and non-irritating material comprising: rubber; plastic; glass; and silicone.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

The words “comprising,” “having,” and “including,” and other forms thereof, are intended to be equivalent in meaning and be open ended in that an item or items following any one of these words is not meant to be an exhaustive listing of such item or items, or meant to be limited to only the listed item or items.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” or “approximately” means within 40.0 mm, 30.0 mm, 20.0 mm, 10.0 mm 5.0 mm 1.0 mm, 0.9 mm, 0.8 mm, 0.7 mm, 0.6 mm, 0.5 mm, 0.4 mm, 0.3 mm, 0.2 mm or 0.1 mm of a given value or range. In certain embodiments, the term “about” or “approximately” means within 20.0 degrees, 15.0 degrees, 10.0 degrees, 9.0 degrees, 8.0 degrees, 7.0 degrees, 6.0 degrees, 5.0 degrees, 4.0 degrees, 3.0 degrees, 2.0 degrees, 1.0 degrees, 0.9 degrees, 0.8 degrees, 0.7 degrees, 0.6 degrees, 0.5 degrees, 0.4 degrees, 0.3 degrees, 0.2 degrees, 0.1 degrees, 0.09 degrees. 0.08 degrees, 0.07 degrees, 0.06 degrees, 0.05 degrees, 0.04 degrees, 0.03 degrees, 0.02 degrees or 0.01 degrees of a given value or range.

As used herein, the terms “connected”, “operationally connected”, “coupled”, “operationally coupled”, “operationally linked”, “operably connected”, “operably coupled”, “operably linked,” and like terms, refer to a relationship (mechanical, linkage, coupling, etc.) between elements whereby operation of one element results in a corresponding, following, or simultaneous operation or actuation of a second element. It is noted that in using said terms to describe inventive embodiments, specific structures or mechanisms that link or couple the elements are typically described. However, unless otherwise specifically stated, when one of said terms is used, the term indicates that the actual linkage or coupling may take a variety of forms, which in certain instances will be readily apparent to a person of ordinary skill in the relevant technology.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An apparatus for non-invasive measurement of intraocular pressure and or ocular durometer of an eye, through an open or closed eyelid of a subject via the sclera, the apparatus comprising:
a frame;
a movement mechanism mounted with respect to the frame;
a support structure mounted with respect to the frame;
a force sensor mounted with respect to the movement mechanism;
a contact tip mounted with respect to the force sensor;
a dipole magnet mounted with respect to the force sensor and the movement mechanism;
a position sensor mounted with respect to the frame;
a microprocessor comprising a memory;
a display unit;
a power source;
wherein the position sensor is configured to track and report the position of the contact tip to the microprocessor,
wherein the support structure holds the eye in place, preventing any movement of the eye during measurement,
wherein the support structure rim compresses the orbital tissues, as to prevent measurement interference from orbital tissues during measurement
wherein the contact tip is placed against the eyelid of the same eye of the subject and moved further towards the eye until a predetermined force threshold is obtained by the force sensor and the correlated position of the contact tip is recorded as the “Zero Point” in the memory, wherein the contact tip is further moved a preset measurement distance towards the eye to obtain a measurement force and the force sensor reports said measurement force to the microprocessor, and
wherein said measurement force, through software instruction, is converted to an intraocular pressure and or ocular durometer, then shown on the display unit and stored in the memory.

2. The apparatus of claim 1, further comprising a communication unit for transmitting said measurement force or intraocular pressure and or ocular durometer to a remote receiver, wherein the communication unit comprises;
a wireless device; and
a wired device.

3. The apparatus of claim 1, further comprising a memory card slot configured for a memory card capable of recording and storing data associated with measurement of the intraocular pressure and or ocular durometer.

4. The apparatus of claim 1, wherein the force sensor comprises:
a strain gauge;
a transducer; and
a load cell.

5. The apparatus of claim 1, wherein the position sensor comprises:
a magnetic position sensor;
an optical position sensor;
a capacitive position sensor; and
a linear variable differential transformer.

6. The apparatus of claim 1, wherein the movement mechanism comprises:
a slide mechanism;
a roller mechanism;
a dovetail rack mechanism;
a rack and pinion mechanism;
a screw mechanism;
a belt-drive mechanism;
a manually driven mechanism;
a motorized mechanism; and
a maglev system.

7. The apparatus of claim 1, further comprising a signaling mechanism which comprises:
an audible signal;
a visual signal; and
a tactile signal.

8. The apparatus of claim 1, wherein the first predetermined force threshold is about ±20% of 1.00 gram.

9. The apparatus of claim 13, wherein the preset measurement distance to obtain a second measurement force is within a range of about ±20% of 0.01 inch.

10. The apparatus of claim 1, wherein the sampling rate for the force sensor by the microprocessor is within a range of about ±20% of 1200 Hz.

11. The apparatus of claim 1, wherein an embodiment used to monitor intraocular pressure and or ocular durometer over a 24 hour period, with preset measurement times, in order to detect diurnal intraocular and or ocular durometer spiking, in the form of:
a night mask;
goggles;
mask;
glasses
head band; and
a helmet

12. The apparatus of claim 1, wherein an embodiment is administered to an animal such as:
a horse;
a dog; and
a cat.

13. The apparatus of claim 1, wherein an embodiment is used to measure tissue comprising:
wound tissue
ocular tissue
organ tissue
vascular tissue
epidermal tissue

14. The apparatus of claim 22, wherein the device is configured for use by an individual, comprising:
a medical clinician;
a medical technician;
a trained individual;
an untrained individual; and
a patient.
is administered to others and or self administered by individual in a setting comprising:
clinical;
home;
ambulatory; and
field combat.

15. The apparatus of claim 9, further comprising a wireless adapter configured to connect with a mobile device and further configured to receive said second measurement force or intraocular pressure and or ocular durometer from the apparatus without a need to pair said apparatus with said mobile device.

16. The apparatus of claim 1, wherein the intraocular pressure and or ocular durometer measurement is not dependent on time.

17. The apparatus of claim 1 wherein the tip is a convex semi-sphere with diameter of less than 0.02 inch, in order to minimize surface area, focusing on reading from eye

18. The apparatus of claim 64 wherein the support structure comprises an eye cup.

19. The apparatus of claim 1, wherein the force sensor requires only one measurement force acquired over one predetermined measurement distance to obtain a suitable intraocular pressure and or ocular durometer measurement.

20. The apparatus of claim 1, wherein the measurement is taken through the eyelid when the eyelid is open or closed.
